(19) United States
(12) Patent Application Publication
(10) Pub. No.: US 2019/0380941 A1
(43) Pub. Date: Dec. 19, 2019

(54) COMPOSITION COMPRISING A TRUFFLE EXTRACT AND NEOHESPERIDIN DIHYDROCHALCONE

(71) Applicant: L'OREAL, Paris (FR)

(72) Inventors: Sonia EYRAUD, Chevilly La Rue (FR); Isabelle BOSSANT, Chevilly La Rue (FR); Beatrice RENAULT, Chevilly La Rue (FR)

(73) Assignee: L'OREAL, Paris (FR)

(21) Appl. No.: 16/464,026

(22) PCT Filed: Nov. 24, 2017
(86) PCT No.: PCT/EP2017/080404

§ 371 Date: May 24, 2019

(30) Foreign Application Priority Data
Nov. 29, 2016 (FR) 1661644

(51) Int. Cl. A61K 8/60; A61K 8/9728; A61Q 19/08

(52) U.S. Cl. CPC A61K 8/602; A61K 2800/805; A61Q 19/08; A61K 8/9728

(57) ABSTRACT

The present application relates to a composition, especially a cosmetic and/or dermatological composition, for topical application, comprising the combination of an aqueous, aqueous-alcoholic or aqueous-glycolic truffle extract with neohesperidin dihydrochalcone (neohesperidin DHC). The present invention also relates to the cosmetic use of said composition, and in particular for the care, hygiene, protection and/or making up of the skin of the body or of the face, or for caring for the hair, preferably for caring for the skin of the body or of the face.

The present application relates to a composition, especially a cosmetic and/or dermatological composition, for topical application, comprising the combination of an aqueous, aqueous-alcoholic or aqueous-glycolic truffle extract with neohesperidin dihydrochalcone (neohesperidin DHC).

The present invention also relates to the cosmetic use of said composition, and in particular for the care, hygiene, protection and/or making up of the skin of the body or of the face, or for caring for the hair, preferably for caring for the skin of the body or of the face.

The present invention relates to the field of ageing and of the signs that are associated therewith, on the skin. It relates in particular to the adjustment of the equilibrium between the proliferation and the differentiation of the epidermal cells.

Women and men currently tend to wish to appear youthful for as long as possible and consequently seek to tone down the signs of ageing on the skin, which are reflected especially by wrinkles and fine lines, thinning of the epidermis and/or a flaccid and withered skin appearance. In this regard, the advertising and fashion industries mention products for retaining radiant and wrinkle-free skin, signs of youthful skin, for as long as possible, all the more so since physical appearance has an effect on the psyche and/or on morale.

The skin constitutes a physical barrier between the body and its surroundings. It is constituted of two tissues: the epidermis and the dermis.

The dermis provides the epidermis with a solid support. It is also its nourishing element. It is mainly constituted of fibroblasts and an extracellular matrix, which is itself composed mainly of collagen, elastin and a substance known as ground substance, these components being synthesized by the fibroblast. Leukocytes, mast cells or else tissue macrophages are also found therein. It also contains blood vessels and nerve fibres.

The epidermis is a desquamating pluristratified epithelium that is 100 μm thick on average and is conventionally divided into a basal layer of keratinocytes that constitutes the germinal layer of the epidermis, a spinous layer constituted of several layers of polyhedral cells positioned on the germinal cells, a granular layer constituted of flattened cells containing distinct cytoplasmic inclusions, keratohyalin granules, and finally an upper layer known as the cornified layer (or stratum corneum), constituted of keratinocytes at the terminal stage of their differentiation, known as corneocytes. These are mummified anucleated cells which derive from keratinocytes. The stack of these corneocytes constitutes the cornified layer that is responsible, inter alia, for the barrier function of the epidermis, i.e. it constitutes a barrier against external attacks, especially chemical, mechanical or infectious attacks and it also makes it possible to protect the body from water loss.

Epidermal differentiation follows a process of continuous and oriented maturation in which the basal keratinocytes transform while migrating so as to result in the formation of corneocytes, dead cells that are completely keratinized. This differentiation is the result of perfectly coordinated phenomena which will result in the thickness of the epidermis being kept constant and thus ensure the homeostasis of the epidermis. This goes through a regulation of the number of cells that enter into the differentiation process and of the number of cells that desquamate. In the course of the normal desquamation process, only the most superficial corneocytes detach from the surface of the epidermis.

Keratins are insoluble proteins produced by the epithelial cells in the form of structurally well organized filaments. These proteins are the main marker of differentiation since throughout epidermal differentiation, various types of keratin will be more or less expressed by the keratinocytes.

Other proteins, associated with keratins, play very important roles in the skin. Filaggrin (or filagrin), a protein present in keratohyalin granules, is produced during the final stages of the differentiation of the epidermis. It is especially involved in the maturation process of the cornified layer by enabling type I and type II keratins to be arranged into coils. This protein thus enables the formation of the cytoplasmic matrix of the surface corneocytes which especially gives the skin its normal thickness, its smooth appearance and its light-reflecting properties. Furthermore, via its degradation within corneocytes, filaggrin provides water-soluble substances having a high osmotic power (natural moisturizing factors or NMFs) that enable good hydration of the cornified layer of the skin to be maintained and thus avoid the feelings of “dry skin”. Filaggrin therefore enables the barrier function of the epidermis to be maintained and makes it possible to avoid drying out the skin.

In the course of chronobiological ageing, the thickness of the epidermis is reduced, maturation of the keratinocytes is imperfect and keratinization no longer leads to an even and homogeneous cornified layer being created. It is also known that prolonged and/or repeated exposures to the sun lead to quite similar results on the epidermis. This is photo-induced ageing. It is also known that, at the menopause, skin ageing accelerates, the thickness of the epidermis decreases, women complain of their skin tightening and that it takes on the look of “dry skin”, or even of the appearance of xerosis.

Surprisingly, the inventors have demonstrated that combining a truffle extract and a suitably selected dihydrochalcone significantly increased filaggrin expression in keratinocytes. As filaggrin is a marker of keratinocyte differentiation, this combination has an effect of stimulating the differentiation of these cells and thus the maturation of the cornified layer. It thus enables the skin to retain or regain a normal thickness, to also have a smooth appearance, that is to say without, or with fewer, wrinkles and fine lines than before its use and a more radiant, less dull complexion.

Furthermore, since filaggrin is involved in skin hydration processes, by increasing the expression of this protein in keratinocytes, the combination of a truffle extract and a suitably selected dihydrochalcone enables the skin to fully carry out its barrier function while avoiding especially the drying out thereof. It therefore makes it possible to re-establish or maintain good hydration of the skin.

The combination of a truffle extract and a suitably selected dihydrochalcone therefore proves particularly advantageous for combating the appearance of the signs of skin ageing and for combating the drying out of the skin, whether or not this is linked to the ageing thereof.

Truffles have been particularly appreciated by epicures since antiquity, due to their extremely rich aroma which gives them a highly characteristic scent and taste.

More recently, truffles have found a use in cosmetics. It is especially known to use a truffle extract for an anti-ageing application. For example, in patent application WO 2016/007461, truffle is described as an agent for reducing the production of reactive oxygen species which may have a deleterious effect on cells, for increasing the production of adenosine triphosphate and for increasing cell viability following an external stress such as exposure to UV radiation. In patent application EP 2 599 492, it is described as an agent for promoting the production of dehydroepiandrosterone (DHEA) which is a hormone which especially plays a role in the process of skin ageing.

Moreover, truffle extracts are known for their anti-inflammatory properties, which enables them to be used in soothing products intended for sensitive skin and as sebum production boosters to compensate the requirements of very dry skin.

Neohesperidin and its derivatives are known for their antioxidant effects. Neohesperidin dihydrochalcone is especially described in patent application FR 2 946 253 in which it is combined with vitamin C, a compound which also has antioxidant properties, to combat ageing associated with the formation of reactive oxygen species in cells.

Within the context of the invention, a synergistic effect has been observed, in a combination of at least one aqueous, aqueous-alcoholic or aqueous-glycolic truffle extract and neohesperidin dihydrochalcone, on filaggrin expression in keratinocytes, and hence on the reduction of the signs of skin ageing and of the drying out of the skin, whether associated or not with the ageing thereof.

Within the meaning of the invention, the effect demonstrated by the combination is termed synergistic in so far as it proves to be greater than that expected by the simple adding together of the respective effects of each of the constituents thereof.

A subject of the invention is therefore a composition for topical application, comprising at least one aqueous, aqueous-alcoholic or aqueous-glycolic truffle extract and neohesperidin dihydrochalcone of the following formula:

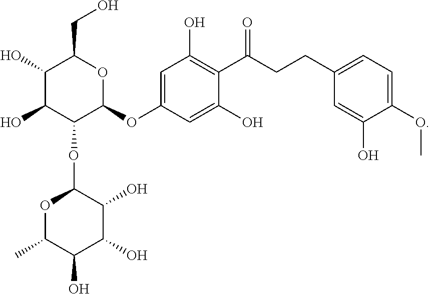 [SMILES: COc1ccc(CCC(=O)c2c(O)cc(O[C@@H]3O[C@H](CO)[C@@H](O)[C@H](O)[C@H]3O[C@@H]3O[C@@H](C)[C@H](O)[C@@H](O)[C@H]3O)cc2O)cc1O]

Another subject of the invention is a non-therapeutic cosmetic process for caring for keratin materials such as the skin, comprising the topical application to these keratin materials of at least one aqueous, aqueous-alcoholic or aqueous-glycolic truffle extract and of neohesperidin dihydrochalcone, or of a composition comprising this combination.

Another subject of the invention is the cosmetic use of said composition, and in particular for the care, protection and/or making up of the skin of the body or of the face, or for caring for the hair, preferably for caring for the skin of the body or of the face.

The term “care” or “caring for” is intended to mean a non-therapeutic care capable of producing an aesthetic effect without, however, preventing or correcting a pathological dysfunction of the skin of the body.

In the following text, the expression “at least one” is equivalent to “one or more” and, unless otherwise indicated, the limits of a range of values are included in that range.

### Truffle Extract

Truffle is the common name given to the edible fruiting body of an ectomycorrhizal ascomyscetes fungus which takes a more or less globular form. The fungus may produce several truffles.

The truffle is the result of the fruiting of a subterranean (hypogeous) fungus. This fruiting body, referred to as the ascocarp, is constituted of the flesh (glebe) and a smooth or warty skin (peridium). The truffle originates from a mycelium (vegetative part of fungi, constituted of fine filaments) which lives in association with the roots of a nourishing tree which may be, for example, an oak, a beech, a hazel, etc., with which it forms symbioses (ectomycorrhiza). It has an antibiotic effect on the vegetation which surrounds the tree.

This association arises by means of mycorrhiza (mixed organ produced by the association of a higher chlorophyll-based plant and of the mycelium of a fungus). Reference is made to mycorrhization, and to controlled mycorrhization when this association results from an inoculation.

A new truffle will originate from mycorrhiza, and will take several months to grow. The period of maturation varies depending on the species of truffle.

The truffle(s) which may be used within the context of the invention are preferentially truffles of the genus Tuber, of the Tuberaceae family, in the Pezizales order. There are more than a hundred species thereof. Among the latter, mention may especially be made of black (Périgord) truffle (Tuber melanosporum), white (Piedmont) truffle (Tuber magnatum), white (summer) truffle (Tuber aestivum), winter truffle (Tuber brumale), bianchetto truffle (Tuber borchii), or else Chinese truffles (Tuber sinensis and Tuber indicum).

According to one particular embodiment, the truffle(s) used within the context of the invention are selected from white truffles and black truffles. According to a preferred embodiment, the truffle(s) used within the context of the invention are black (Périgord) truffle (Tuber melanosporum), white (summer) truffle (Tuber aestivum), or a mixture of the two.

Within the context of the invention, the truffle extract(s) may especially be obtained from an extraction solvent by using an extraction technique selected from the extraction techniques well known from the prior art. Within the meaning of the present invention, extraction is intended to mean a process which makes it possible to obtain a chemical species from a natural substance containing same. Among the traditional extraction techniques, mention may especially be made of filtration, pressing, decoction, enfleurage, percolation, infusion, maceration, steam entrainment or hydrodistillation, soxhlet extraction, batch-stirred extraction, sonication-assisted extraction, microwave-assisted extraction, accelerated solvent extraction, subcritical or supercritical fluid extraction.

Generally, the extraction solvent is chosen from water, water-soluble or water-miscible solvents (hydrophilic solvents), and mixtures thereof.

Among the hydrophilic solvents, mention may especially be made of substantially linear or branched lower monoalcohols having from 1 to 8 carbon atoms, such as ethanol, propanol, butanol, isopropanol or isobutanol; polyols, such as propylene glycol, isoprene glycol, butylene glycol, propylene glycol, glycerol, sorbitol, polyethylene glycols and derivatives thereof; and mixtures thereof.

When the extraction solvent is water, the truffle extract is said to be aqueous.

When the extraction solvent is a substantially linear or branched lower monoalcohol having from 1 to 8 carbon atoms, the truffle extract is said to be alcoholic.

When the extraction solvent is a polyol, the truffle extract is said to be glycolic.

When the extraction solvent is a mixture of water and of one or more substantially linear or branched lower monoalcohols having from 1 to 8 carbon atoms, the extract is said to be aqueous-alcoholic.

When the extraction solvent is a mixture of water and of one or more polyols, the extract is said to be aqueous-glycolic.

Within the context of the invention, the truffle extract(s) are obtained from an extraction solvent comprising water. The truffle extract(s) are then aqueous, aqueous-alcoholic or aqueous-glycolic. Preferably, the truffle extract(s) are aqueous or aqueous-glycolic.

According to a particular embodiment, the truffle extract(s) are obtained by microwave-assisted extraction, the extraction solvent being water, by steam entrainment, or by extraction with a water/glycerol mixture.

According to a first embodiment, the truffle extract is obtained by microwave-assisted extraction, the extraction solvent being water. According to a preferred mode, after a filtration step, a propylene glycol/water mixture is added to the aqueous extract obtained in this way. Even more preferentially, this is a white (summer) truffle extract (Tuber aestivum). By way of example, mention may be made of the product White Truffle Extract PG sold by Crodarom which is an aqueous extract obtained by microwave-assisted extraction dissolved in a propylene glycol/water mixture, this product thus comprising 1.53% by weight of active material of white (summer) truffle (Tuber aestivum) in a propylene glycol/water (75.50/22.96) mixture.

According to a second embodiment, the truffle extract is obtained by steam entrainment. This is preferably a black (Périgord) truffle (Tuber melanosporum) extract. By way of example, mention may be made of the product Végébios® NAT of Black Truffle sold by Solabia, which is an aqueous extract containing 0.5% by weight of active material of black (Périgord) truffle (Tuber melanosporum) in water.

According to a third embodiment, the truffle extract is obtained by extraction with a water/glycerol mixture. This is preferably an extract of black (Périgord) truffle (Tuber melanosporum). By way of example, mention may be made of the product Glycerolat® HG of Black Truffle sold by Solabia which is an extract comprising 0.5% by weight active material of black (Périgord) truffle (Tuber melanosporum) in a glycerol/water (50/48.8) mixture.

According to a particular embodiment of the invention, the truffle extract(s) are present in the composition in an amount of raw material ranging from 0.001% to 5% by weight, preferably from 0.01% to 1% by weight, and even more preferentially from 0.01% to 0.5% by weight relative to the total weight of the composition.

According to a particular embodiment of the invention, the truffle extract(s) are present in the composition in an amount of active material ranging from 1·10⁻⁵% to 1% by weight, preferably from 1·10⁻⁴% to 0.1% by weight, and even more preferentially from 1·10⁻⁴% to 0.01% by weight relative to the total weight of the composition.

### Neohesperidin Dihydrochalcone

Neohesperidin dihydrochalcone (DHC) is a polyphenol of natural oxygen with a large antioxidant potential over a very broad spectrum of several species of radicals, which is capable of acting on three cellular targets: membrane, nucleus and cytoplasm.

Neohesperidin dihydrochalcone (DHC) is part of the dihydrochalcones family, which is part of the flavonoids class, pigments which are virtually universal in plants. Neohesperidin DHC is a glycosylated flavonoid which has the following structure:

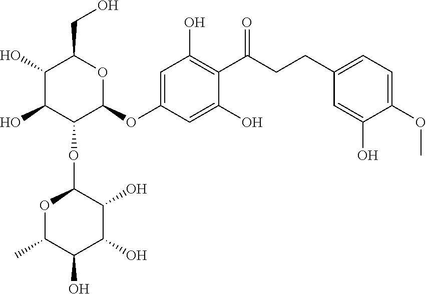 [SMILES: COc1ccc(CCC(=O)c2c(O)cc(O[C@@H]3O[C@H](CO)[C@@H](O)[C@H](O)[C@H]3O[C@@H]3O[C@@H](C)[C@H](O)[C@@H](O)[C@H]3O)cc2O)cc1O]

It is also known under its IUPAC name: 1-(4-((2-O-[6-Deoxy-α-L-mannopyranosyl]-β-D-glucopyranosyl)oxy)-2,6-dihydroxyphenyl)-3-[3-hydroxy-4-methoxyphenyl]-1-propanone.

Neohesperidin DHC (CAS number 20702-77-6) may especially be obtained either from neohesperidin which may be extracted from bitter orange (Citrus aurantium) or from naringin which is obtained from grapefruit (Citrus paradisii). Synthesis from extracted neohesperidin involves hydrogenation in the presence of a catalyst under alkaline conditions. Synthesis from naringin is based on the conversion thereof to give phloroacetophenone-4′-β-neohesperidoside, which may be condensed with isovanillin (3-hydroxy-4-methoxybenzaldehyde) to produce neohesperidin (see the following references: Borrego, F. Sweeteners (3rd edition), 2007, 67-77 and Borrego, F; Montijano, H. Food Science and Technology, 2001, 112 (Alternatives Sweeteners), 87-104).

Neohesperidin dihydrochalcone is especially available under the commercial reference Neohesperidin DC from Ferrer (Zoster).

Neohesperidin dihydrochalcone may exist in the hydrate form.

According to a particular embodiment, the neohesperidin dihydrochalcone is present in the composition according to the invention in an amount of active material ranging from 0.01% to 10% by weight, preferably from 0.01% to 1% by weight and even more preferentially from 0.01% to 0.2% by weight, relative to the total weight of the composition.

According to a particular embodiment of the invention, the weight ratio of neohesperidin dihydrochalcone to the truffle extracts is between 10/1 and 1/100, preferably 1/1 and 1/10, and even more preferentially 1/1 and 1/5.

“Cutaneous signs of ageing” or “signs of skin ageing” is intended to mean any modifications in the external appearance of the skin due to ageing, whether chronobiological and/or photoinduced, such as, for example, wrinkles and fine lines, withered skin, flaccid skin, slack skin, thinned skin, dry skin, dull skin that lacks radiance, heterogeneity of the complexion and of the surface of the skin. The signs of chronobiological or chronological ageing (also known as chronoageing) correspond to internal degradations of the skin due to the intrinsic ageing of individuals. The signs of photoinduced ageing (or photoageing) correspond to internal degradations of the skin following exposure to ultraviolet radiation.

“Skin” is intended to denote, for the purposes of the invention, the whole of the body covering, and especially the skin, mucous membranes and scalp.

The reduction of the signs of skin ageing and/or the delaying of their appearance, via the use of the combination according to the present invention, occurs in particular owing to the increase or improvement in the differentiation of the epidermal cells and/or the increase or stimulation especially of filaggrin expression in epidermal keratinocytes.

Preferably, the compositions used according to the invention are cosmetic compositions, i.e. they are intended to improve the aesthetic appearance of the individual.

The use according to the present invention is especially effective for combating the signs, in particular aesthetic signs, of chronobiological and/or photoinduced ageing of the epidermis. Through the present invention, combating the signs of chronobiological ageing of the skin will preferentially be targeted.

The present invention is thus effective in any person regardless of their age. When combating the signs of chronobiological ageing, the individuals preferentially targeted will be individuals more than 30 years old, preferentially more than 40 years old.

The signs of skin ageing are preferentially chosen from the appearance of wrinkles and fine lines and/or the weakening and/or the slackening and/or the withering and/or the thinning and/or dryness and/or the dull and/or lacklustre appearance and/or the complexion and/or the heterogeneous surface of the skin.

Through the present invention, skin having a more youthful appearance and being better hydrated is therefore obtained.

A composition in accordance with the invention, namely intended for the implementation of the invention, may be a cosmetic or dermatological composition according to the application envisaged, and therefore comprises a physiologically acceptable medium.

For the purposes of the present invention, “physiologically acceptable medium” is intended to mean a medium suitable for the topical administration of a composition, and compatible with all the keratin materials, such as the skin, the scalp, the nails, the mucous membranes, the eyes and the keratin fibres such as the hair or eyelashes, or any other area of bodily skin.

A physiologically acceptable medium can be a dermatologically or cosmetically acceptable medium; it is preferentially a cosmetically acceptable medium, i.e. devoid of any unpleasant appearance or odour, and which is entirely compatible with the topical administration route. In the present case, the composition is intended to be administered topically, i.e. by application at the surface of the keratin material under consideration, and more particularly of the skin under consideration.

The cosmetic or dermatological compositions capable of being used in the context of the invention generally comprise a physiologically acceptable medium, preferably a cosmetically acceptable medium.

The compositions according to the invention may be in all the galenical forms conventionally used for a topical application and especially in the form of aqueous or aqueous-alcoholic solutions, of oil-in-water (O/W), water-in-oil (W/O) or multiple (triple: W/O/W or O/W/O) emulsions, of aqueous gels or of dispersions of a fatty phase in an aqueous phase using spherules, it being possible for these spherules to be lipid vesicles of ionic and/or non-ionic type (liposomes, niosomes or oleosomes). These compositions are prepared according to the usual methods.

The compositions according to the invention may comprise at least one aqueous phase.

The aqueous phase contains water and optionally other water-soluble or water-miscible organic solvents.

An aqueous phase that is suitable for use in the invention may comprise, for example, a water chosen from a natural spring water, such as water from La Roche-Posay, water from Vittel or waters from Vichy, or a floral water.

The aqueous phase may comprise at least one hydrophilic solvent, such as, for example, substantially linear or branched lower monoalcohols having from 1 to 8 carbon atoms, such as ethanol, propanol, butanol, isopropanol or isobutanol; polyols, such as propylene glycol, isoprene glycol, butylene glycol, glycerol, sorbitol, polyethylene glycols and derivatives thereof; and mixtures thereof.

According to the galenical form of the composition, the amount of aqueous phase may range from 0.1% to 99% by weight, preferably from 0.5% to 98% by weight, better still from 30 to 95% by weight and even better still from 40 to 95% by weight relative to the total weight of the composition.

According to a particular embodiment, the compositions in accordance with the invention comprise an aqueous phase. According to a preferred embodiment, the compositions in accordance with the invention are aqueous or aqueous-alcoholic solutions.

The compositions according to the invention may also be in anhydrous form, such as, for example, in the form of an oil. “Anhydrous composition” is intended to mean a composition containing less than 1% by weight of water, or even less than 0.5% of water, and especially free of water, the water not being added during the preparation of the composition but corresponding to the residual water provided by the mixed ingredients.

Advantageously, the compositions according to the invention are in the form of a gel, of an emulsion, of a powder or of a paste.

In addition, the composition according to the invention may be more or less fluid and may have the appearance of a white or coloured cream, an ointment, a milk, a lotion, a serum, a paste, a foaming gel, a care product, a tonic or a foam. It may optionally be applied to the skin in aerosol form. It may also be in solid form, and for example in the form of a stick.

When the composition used according to the invention comprises an oily phase, it preferably contains at least one oil. It may also contain other fatty substances.

As oils that may be used in the composition of the invention, mention may be made, for example, of:
hydrocarbon-based oils of animal origin, such as perhydrosqualene;
hydrocarbon-based oils of plant origin, such as liquid fatty acid triglycerides comprising from 4 to 10 carbon atoms, such as heptanoic or octanoic acid triglycerides, or else, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides, such as those sold by Stéarineries Dubois or those sold under the names Miglyol 810 N and Miglyol 818 by Sasol, and Miglyol 812 N by Cremer Oleo, jojoba oil and shea butter oil;
synthetic esters and ethers, especially of fatty acids, such as the oils of formulae R′COOR² and R′OR² in which R′ represents the residue of a fatty acid comprising from 8 to 29 carbon atoms, and R² represents a branched or unbranched hydrocarbon-based chain containing from 3 to 30 carbon atoms, such as, for example, Purcellin oil, isononyl isononanoate, isopropyl myristate, 2-ethylhexyl palmitate, 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate or triisocetyl citrate; fatty alcohol heptanoates, octanoates or decanoates; polyol esters, such as propylene glycol dioctanoate, neopentyl glycol diheptanoate and diethylene glycol diisononanoate; and pentaerythritol esters, such as pentaerythrityl tetraisostearate;
linear or branched hydrocarbons of inorganic or synthetic origin, such as volatile or non-volatile liquid paraffins, and derivatives thereof, petroleum jelly, polydecenes, and hydrogenated polyisobutene such as Parleam oil;
fatty alcohols having from 8 to 26 carbon atoms, such as cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils, such as those described in document JP-A-2-295 912;
silicone oils, such as volatile or non-volatile polydimethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, groups having from 2 to 24 carbon atoms; phenylsilicones, such as phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes;
mixtures thereof.

In the list of the abovementioned oils, the term “hydrocarbon-based oil” is intended to mean any oil predominantly comprising carbon and hydrogen atoms, and optionally ester, ether, fluoro, carboxylic acid and/or alcohol groups.

The other fatty substances that may be present in the oily phase are, for example, fatty acids comprising from 8 to 30 carbon atoms, such as stearic acid, lauric acid, palmitic acid and oleic acid; waxes, such as lanolin wax, beeswax, carnauba wax or candelilla wax, paraffin waxes, lignite wax or microcrystalline waxes, ceresin or ozokerite, and synthetic waxes, such as polyethylene waxes and Fischer-Tropsch waxes; silicone resins such as trifluoromethyl-C₁-C₄-alkyl dimethicone and trifluoropropyl dimethicone; and silicone elastomers, such as the products sold under the name KSG by the company Shin-Etsu, under the name Trefil, BY29 or EPSX by the company Dow Corning, or under the name Gransil by the company Grant Industries.

These fatty substances can be chosen in a varied way by those skilled in the art so as to prepare a composition having the desired properties, for example of consistency or texture. According to one particular embodiment of the invention, the composition according to the invention is a water-in-oil (W/O) or oil-in-water (O/W) emulsion. The proportion of the oily phase of the emulsion may range from 5 to 90% by weight and preferably from 5 to 60% by weight relative to the total weight of the composition.

The emulsions generally contain at least one emulsifier chosen from amphoteric, anionic, cationic and non-ionic emulsifiers, used alone or as a mixture, and optionally a co-emulsifier. The emulsifiers are chosen in an appropriate manner according to the emulsion to be obtained (W/0 or O/W emulsion). The emulsifier and the co-emulsifier are generally present in the composition in a proportion ranging from 0.3% to 30% by weight and preferably from 0.5% to 20% by weight relative to the total weight of the composition.

For W/O emulsions, examples of emulsifiers that may be mentioned include dimethicone copolyols, such as the mixture of cyclomethicone and dimethicone copolyol sold under the name DC 5225 C by Dow Corning, and alkyl dimethicone copolyols such as the lauryl methicone copolyol sold under the name Dow Corning 5200 Formulation Aid by Dow Corning, and the cetyl dimethicone copolyol sold under the name Abil EM 90® by Goldschmidt. A crosslinked elastomeric solid organopolysiloxane comprising at least one oxyalkylenated group, such as those obtained according to the procedure of Examples 3, 4 and 8 of document U.S. Pat. No. 5,412,004 and of the examples of document U.S. Pat. No. 5,811,487, especially the product of Example 3 (synthesis example) of patent U.S. Pat. No. 5,412,004, such as the product sold under the reference KSG 210 by the company Shin-Etsu, may also be used as surfactants for W/0 emulsions.

For the O/W emulsions, examples of emulsifiers that may be mentioned include non-ionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-40 stearate.

The composition according to the invention may also contain adjuvants which are customary in the cosmetics field, such as hydrophilic or lipophilic gelling agents, preservatives, water, solvents, fragrances, fillers, waxes, pasty fatty substances, sunscreens or UV-screening agents, odour absorbers, colorants, basic agents, acids, sequestrants, polyols, or non-ionic, anionic or cationic surfactants.

The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

The compositions according to the invention may be applied directly to the skin or, alternatively, to cosmetic supports of occlusive or non-occlusive type, intended to be applied locally to the skin. By way of non-limiting examples of cosmetic supports, mention may in particular be made of a patch, a wipe, a roll-on and a pen.

The compositions of the invention may contain additional active agents chosen from antioxidants other than the truffle extracts and the neohesperidin DHC as defined above, dermo-relaxing or dermo-decontracting agents, anti-ageing agents, moisturizing agents, chelating agents, vitamins, depigmenting agents, anti-glycation agents, agents for stimulating the synthesis of dermal or epidermal macromolecules and/or for preventing their degradation, agents for stimulating fibroblast or keratinocyte proliferation and/or keratinocyte differentiation, agents for promoting maturation of the cornified envelope, NO-synthase inhibitors, agents for stimulating the energy metabolism of cells, and desquamating agents.

The additional active agent for caring for keratin materials, such as the skin, that is used in the composition according to the invention may represent from 0.0001% to 20%, preferably from 0.01% to 10% and better still from 0.01% to 5% by weight relative to the total weight of the composition.

The cosmetic composition may optionally be rinsed after having been applied to the skin. Moreover, after the application of the cosmetic composition according to the invention, a composition comprising one or more active agents chosen from antibacterial agents, antifungal agents and/or powders may be applied to the surface of the skin.

According to one particular embodiment of the invention, other agents intended to make the appearance and/or the texture of the skin more attractive may also be added to the composition suitable for use in the invention.

Needless to say, a person skilled in the art will take care to select the optional adjuvant(s) added to the composition according to the invention such that the advantageous properties intrinsically associated with the composition in accordance with the invention are not, or are not substantially, adversely affected by the envisioned addition.

The examples that follow will allow the invention to be understood more clearly, without, however, being limiting in nature. The amounts indicated are weight percentages of raw materials, unless otherwise mentioned. The names of the compounds are given as the INCI names.

### EXAMPLES

### Example 1: Demonstration of the Activity of the Combinations According to the Invention

The in vitro effect of the combination of neohesperidin dihydrochalcone (neohesperidin DHC) and an extract of black (Périgord) truffle (Tuber melanosporum) and/or an extract of white (summer) truffle (Tuber aestivum) on keratinocyte differentiation is studied. The expression and the location of the marker of filaggrin differentiation are especially studied in keratinocytes in culture which thus makes it possible to evaluate the ability of these combinations to increase the differentiation of these cells.

### Procedure

### Culture Medium and Test Medium

The culture medium is:

Keratinocyte-SFM supplemented with:
0.25 ng/ml Epidermal Growth Factor (EGF);
25 μg/ml pituitary extract (PE);
25 μg/ml Gentamicin;

The test medium is Keratinocyte-SFM supplemented with 25 μg/ml Gentamicin.

### Culture and Treatment:

The keratinocytes were seeded into 96-well plates and cultured in culture medium for 168 hours with renewal of the culture medium after 24 and 96 hours of incubation. The culture medium was then replaced with the test medium alone (control) or containing one of the compounds or one of the combinations of each of its compounds to be tested, or the reference (CaCl₂)), then the cells were incubated for 72 hours.

The reference used is a solution of calcium chloride. Since calcium chloride is known as a stimulator of keratinocyte differentiation, it was used as reference molecule for this test.

All the experimental conditions were carried out in n=3, except the control condition which was carried out in n=6.

### Expression of the Proteins in Normal Human Keratinocytes (NHEK)—In-Situ Immunolabelling

After incubation, the medium was eliminated and the cells were rinsed, fixed and permeabilized. The cells were then labelled with the primary antibody (see Table A) directed against the protein of interest (filaggrin). This antibody was then revealed by a secondary antibody coupled to a fluorochrome (see Table A). At the same time, the nuclei of the cells were stained with Hoechst 33258 (bisbenzimide).

TABLE A
Primary and secondary antibody

| Protein | Primary antibody | Secondary antibody |
|---|---|---|
| Filaggrin | Anti filaggrin Santa Cruz, ref. | GAM-Alexa 488 Molecular |
| | SC-66192 | Probes, ref. A11001 |

The image acquisition was carried out with an INCellAnalyzer™ 1000 (GE Healthcare) high-resolution imaging system. For each well, 10 digitized images were captured for each immunolabelling (×20 objective).

The labellings were qualified by measuring the fluorescence intensity of the proteins relative to the number of cells identified by the Hoechst product (integration of the digital data using the Developer Toolbox 1.5 software, GE Healthcare).

### Results

The results are given in the table below.

| | Concentration | | | |
|---|---|---|---|---|
| | (by weight of | | | |
| | active material, | Mean | % relative | |
| | unless indicated | filaggrin | to the | |
| Compounds tested | otherwise) | (AU) | control | P ⁽¹⁾ |
| Control | | 12261 | 100 | — |
| CaCl₂ (positive con- | 1.5 mM | 74795 | 610 | *** |
| trol for validating | | | | |
| the study) | | | | |
| Black truffle extract | 4.5 · 10⁻³% | 15272 | 125 | ns |
| White truffle extract | 4.5 · 10⁻³% | 17993 | 147 | ** |
| Neohesperidin DHC | 0.3 mg/ml | 18840 | 154 | * |
| Black truffle extract + | 4.5 · 10⁻³% + | 26825 | 219 | *** |
| Neohesperidin DHC | 0.3 mg/ml | | | |
| White truffle extract + | 4.5 · 10⁻³% + | 31187 | 254 | *** |
| Neohesperidin DHC | 0.3 mg/ml | | | |
| Black truffle extract + | 4.5 · 10⁻³% + | 29310 | 239 | *** |
| white truffle extract + | 4.5 · 10⁻³% + | | | |
| Neohesperidin DHC | 0.3 mg/ml | | | |

⁽¹⁾ P: threshold for statistical significance
NS > 0.05, not significant
0.01 to 0.05% significant
** 0.001 to 0.01, very significant
*** <0.001, extremely significant

Under the experimental conditions of this study, the treatment with calcium chloride, pro-differentiating reference molecule, substantially increases the expression of filaggrin by the keratinocytes (+510% relative to the control).
The white truffle and the neohesperidin DHC tested alone significantly increase the filaggrin protein expression: the stimulatory effect of the neohesperidin DHC (+54%) is greater than that of the white truffle (+47%).
The black truffle tested alone increases the filaggrin protein expression non-significantly (+25%).
When the neohesperidin DHC is tested in combination with the white truffle and/or the black truffle, a significantly better effect is noted than those observed after a treatment with the active agents tested alone (neohesperidin+white truffle: +154%; neohesperidin DHC+black truffle: +119%; neohesperidin DHC+white truffle+black truffle: +139%).

### Conclusion

Thus, the combinations of neohesperidin DHC with a black truffle extract and/or with a white truffle extract according to the invention significantly increase the filaggrin protein expression in normal human epidermal keratinocytes.

The results obtained thus translate into an increase in epidermal differentiation. The combinations of neohesperidin DHC with a black truffle extract and/or a white truffle extract according to the invention therefore have a pro-differentiating effect on normal human keratinocytes and may thus decrease and/or delay the signs of skin ageing.

A synergy between neohesperidin DHC and the truffle extract(s) has been observed, the effects obtained with the different combinations tested being greater than the sum of the effects obtained for each of the compounds of the combination tested alone at the same concentration as in the combination.

### Example 2: Cosmetic Compositions in the Form of a Direct Emulsion

The following compositions are produced.

| Composition | A | B |
|---|---|---|
| PEG-100 stearate (Croda - MYRJ 100-PA-(SG)) | 0.3% | — |
| CETEARYL ALCOHOL (and) CETEARYL | 0.32% | — |
| GLUCOSIDE (SEPPIC - MONTANOV 68) | | |
| Screening agent(s) | 20% | — |
| PHENOXYETHANOL | 0.7% | 0.7% |
| NEOHESPERIDIN DIHYDROCHALCONE | 0.02% | 0.02% |
| (ZOSTER (FERRER) - NEOHESPERIDINE | | |
| DC PH EUR) | | |
| GLYCEROL | 7% | 15% |
| ISOPROPYL ISOSTEARATE | 2% | — |
| POLYACRYLAMIDE (and) C13-14 | 1.5% | — |
| ISOPARAFFIN (and) LAURETH-7 | | |
| (SEPPIC - SEPIGEL 305) | | |
| DIMETHICONE (and) DIMETHICONOL | 5% | — |
| (DOW CORNING - XIAMETER PMX-1503 | | |
| FLUID) | | |
| TUBER MELANOSPORUM EXTRACT | 0.1% | 0.1% |
| (SOLABIA - VEGEBIOS NAT OF | | |
| BLACK TRUFFLE) | | |
| TUBER AESTIVUM EXTRACT | 0.1% | 0.1% |
| (CRODAROM - WHITE TRUFFLE | | |
| EXTRACT PG) | | |
| WATER | qs | qs |
| DIMETHICONE (DOW CORNING - DOW | — | 1% |
| CORNING TORAY SH 200 C FLUID 10 CS) | | |
| PEG-20 METHYL GLUCOSE | — | 1% |
| SESQUISTEARATE (LUBRIZOL - | | |
| GLUCAMATE SSE-20 EMULSIFIER) | | |
| LIMNANTHES ALBA SEED OIL (NATURAL | — | 5% |
| PLANT PRODUCTS - MEADOWFOAM SEED | | |
| OIL) | | |
| AMMONIUM POLYACRYLOYLDIMETHYL | — | 0.9% |
| TAURATE (CLARIANT - HOSTACERIN | | |
| AMPS) | | |
| pH adjusters | qs pH 5.5 | qs pH 5.5 |
| Alcohol | 2% | 5% |

### Preparation Method

Heat the fatty phase to 65° C.

Heat the aqueous phase to 65° C.

Pour the fatty phase gently over the aqueous phase. At around 40° C., gel the mixture.

Prepare a phase with neohesperidin DHC and 15% water, heat this phase to 50° C., disperse, homogenize.

Add the active agents (truffle extracts and phase containing neohesperidin DHC) when the mixture is at around 30° C., then add the alcohol.

The compositions A and B according to the invention, when they are applied daily to the face, make it possible to combat the signs of skin ageing.

### Example 3: Cosmetic Composition in the Form of an Aqueous Gel

The following composition is produced.

| Composition | C |
|---|---|
| NEOHESPERIDIN DIHYDROCHALCONE (ZOSTER | 0.04% |
| (FERRER) - NEOHESPERIDINE DC PH EUR) | |
| Glycerol | 10% |
| BIOSACCHARIDE GUM-1 (SOLABIA - FUCOGEL 1.5P) | 6% |
| TUBER MELANOSPORUM EXTRACT (SOLABIA - | 0.2% |
| VEGEBIOS NAT OF BLACK TRUFFLE) | |
| TUBER AESTIVUM EXTRACT (CRODAROM - WHITE | 0.2% |
| TRUFFLE EXTRACT PG) | |
| AMMONIUM POLYACRYLOYLDIMETHYL TAURATE | 0.8% |
| (CLARIANT - HOSTACERIN AMPS) | |
| ISOSTEARYL | 0.7% |
| Alcohol | 4% |
| WATER | qs |

### Preparation Method

In a beaker, add water, glycerol, neohesperidin DHC and phenoxyethanol. Heat the mixture to 65° C. Homogenize. Cool to around 40° C., add the gelling agents AMPS and fucogel. At around 30° C., add the truffle extracts. Then add the alcohol.

The composition C according to the invention, when it is applied daily to the face, makes it possible to combat the signs of skin ageing.

### Example 4: Cosmetic Composition in the Form of an Emulsified Gel

The following composition is produced.

| Composition | D |
|---|---|
| NEOHESPERIDIN DIHYDROCHALCONE (ZOSTER | 0.04% |
| (FERRER) - NEOHESPERIDINE DC PH EUR) | |
| GLYCEROL | 10% |
| BIOSACCHARIDE GUM-1 (SOLABIA - FUCOGEL 1.5P) | 6% |
| SOHEXADECANE (CRODA - ARLAMOL HD) | 2% |
| CYCLOHEXASILOXANE (SHIN ETSU - KF-996) | 4% |
| TUBER MELANOSPORUM EXTRACT (SOLABIA - | 0.2% |
| VEGEBIOS NAT OF BLACK TRUFFLE) | |
| TUBER AESTIVUM EXTRACT (CRODAROM - WHITE | 0.2% |
| TRUFFLE EXTRACT PG) | |
| AMMONIUM POLYACRYLOYLDIMETHYL TAURATE | 0.8% |
| (CLARIANT - HOSTACERIN AMPS) | |
| ISOSTEARYL | 0.7% |
| Alcohol | 4% |
| WATER | qs |

### Preparation Method

Heat the aqueous phase to 50° C. with neohesperidin DHC. Heat the fatty phase to about 50° C. Emulsification: pour the fatty phase over the aqueous phase. Gel the mixture at around 40° C. Add the active agents (truffle extracts) at around 30-35° C. Add the alcohol at around 30° C.

The composition D according to the invention, when it is applied daily to the face, makes it possible to combat the signs of skin ageing.

1. A composition, comprising:
at least one aqueous, aqueous-alcoholic or aqueous-glycolic truffle extract and neohesperidin dihydrochalcone having a formula:

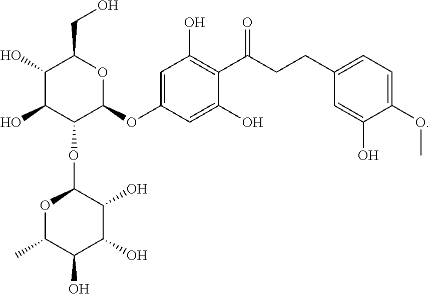 [SMILES: COc1ccc(CCC(=O)c2c(O)cc(O[C@@H]3O[C@H](CO)[C@@H](O)[C@H](O)[C@H]3O[C@@H]3O[C@@H](C)[C@H](O)[C@@H](O)[C@H]3O)cc2O)cc1O]

2. The composition according to claim 1,
wherein the neohesperidin dihydrochalcone is present in an amount of active material ranging from 0.01% to 10% by weight, relative to the total weight of the composition.

3. The composition according to claim 1, wherein the truffle extract(s) are aqueous or aqueous-alcoholic.

4. The composition according to claim 1, wherein the truffle extract(s) are obtained by microwave-assisted extraction, the extraction solvent being water, by steam entrainment, or by extraction with a water/glycerol mixture.

5. The composition according to claim 1, wherein the truffle(s) are selected from white truffles, black truffles and mixtures thereof.

6. The composition according to claim 1, wherein the truffle extract(s) are present in an amount of active material ranging from 1·10⁻⁵% to 1% by weight relative to the total weight of the composition.

7. The composition according to claim 1, wherein the weight ratio of neohesperidin dihydrochalcone to truffle extracts is between 10/1 and 1/100.

8. A non-therapeutic cosmetic treatment process for caring for keratin materials, comprising the topical application to the keratin materials of a composition as defined in claim 1.

9. The process according to claim 8 for combating the signs of chronobiological and/or photo-induced ageing of the epidermis.

10. The process according to claim 8 for increasing the softness of the skin and/or the radiance of the skin.

11. A process for caring for, protecting and/or making up the skin of the body or of the face, or for caring for the hair, comprising applying the composition according to claim 1.

12. The process according to claim 11 for combating the signs of chronobiological and/or photo-induced ageing of the epidermis.

13. The process according to claim 11 for increasing the softness of the skin and/or the radiance of the skin.
